(19) United States
(12) Patent Application Publication
(10) Pub. No.: US 2019/0117153 A1
(43) Pub. Date: Apr. 25, 2019

(54) METHODS FOR MONITORING BLADDER CANCER IMMUNOTHERAPY

(71) Applicant: NantBio, Inc., Culver City, CA (US)

(72) Inventors: Patrick Soon-Shiong, Culver City, CA (US); Kayvan Niazi, Encino, CA (US)

(21) Appl. No.: 16/142,794

(22) Filed: Sep. 26, 2018

Related U.S. Application Data
(60) Provisional application No. 62/574,822, filed on Oct. 20, 2017.

(51) Int. Cl. A61B 5/00; A61K 49/00; A61K 35/17; A61B 1/307; A61B 1/04; A61M 31/00; A61B 5/20; A61B 1/06

(52) U.S. Cl. CPC A61B 5/4848; A61K 49/006; A61K 35/17; A61B 1/307; A61B 5/4842; A61M 2210/1085; A61M 31/005; A61B 5/202; A61B 1/063; A61B 1/0653; A61B 1/043

(57) ABSTRACT

The invention provides methods of measuring the progression and effectiveness of a course of treatment of bladder cancer in a subject diagnosed with a bladder cancer, by applying a physiologically acceptable dye to the tumor and measuring the degree of progression and effectiveness of the course of treatment of the bladder cancer.

### CROSS REFERENCE TO RELATED APPLICATIONS

This application claims the benefit of priority from U.S. Provisional Application No. 62/574,822, filed on Oct. 20, 2017, which is incorporated by reference herein.

### FIELD OF THE INVENTION

The present disclosure relates generally to methods for treating bladder cancers by immunotherapy and to methods of monitoring the progress of the treatment in a subject needing such treatment

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Bladder cancer is a type of cancer that originates from cells of the urinary bladder. More than 90% of bladder cancers originate as transitional cell carcinomas, arising from the urotheleum. The urotheleum is the epithelial layer of the inner lining of the bladder. Other varieties of cancer found in the bladder include squamous cell carcinomas, adenocarcinomas, sarcomas and small cell carcinomas. Diagnosis and treatment of bladder cancer depends, to a great degree, on the stage at which the cancer is discovered. As summarized by U.S. Pat. No. 9,523,689, bladder cancer can be staged by the Tumor-Node-Metastases (TNM) classification (American Joint Committee on Cancer). In the TNM system, bladder cancer tumors are sorted by particular properties. For example, invasive tumors that are not in muscle, such as papillary tumors that are confined to the epithelial mucosa, are defined as Ta tumors. In another example, tumors that invade the subepithelial tissue (i.e., lamina propria) are defined as T1 tumors. Tumors with a distinct morphology and a dynamic phenotype are considered to be carcinoma in situ (Tis). Invasive tumors (T2-4a and T2-4b) are further sorted based on the degree of their invasive appearance as revealed by histopathological testing. Thus, a T2 tumor has penetrated into the muscle layer. A T3 tumor has penetrated into the fatty tissue surrounding the bladder, and a T4 tumor has grown to reach the pelvic or abdominal wall.

One of the most effective tools for detecting and diagnosing early bladder cancer is the cystoscope. The cystoscope is the product of at least two centuries of development, according to Samplaski and Jones, 2009 (BJU Int. 103(2):154-8). The modern cystoscope is an endoscope with a rigid or flexible tube, with a light and camera, allowing for visual inspection of the lining of the urethra and bladder and optionally, surgical intervention or tissue sampling through the urethra. While other imaging and detection technologies for diagnosing and monitoring cancers are now available, the cystoscope has many advantages. For example, the cystoscope provides direct visualization of the lining of the bladder, and allows for transurethral biopsy sampling or surgical removal of superficial tumors that are visible on or adjacent to the urotheleum.

A number of methods have been used to assist in visualizing tumor tissue present in or on the lining of the bladder. One of these is selective staining surface staining of bladder cancer with methylene blue, as described, for example, by Gil et al., 1984, (“Gil,” Cancer, 53: 2124-2127).

U.S. Pat. No. 5,301,688 describes intravesical electromotive administration of dyes to provide differential staining of cancerous and normal urothelium. The '688 patent employs an electrical gradient to actively transport dyes, such as methylene blue, into the tumor cells.

U.S. Pat. No. 6,083,487 describes intravesical staining of bladder tumors with methylene blue or toluidine blue as photosensitizers, followed by laser illumination of the bladder wall at a wavelength, e.g., 630 or 660 nm., that is appropriate to induce fluorescence in the tumor tissue bound methylene blue or toluidine blue dye.

There is also a porphyrin based commercial system designed to enhance detection of bladder cancer, in particular carcinoma in situ (CIS). For example, see U.S. Pat. No. 7,850,008. Cysview® (hexaminolevulinate HCl; Hexvix® in Europe) is an optical imaging agent licensed by Ipsen from Photocure ASA. Hexaminolevulinate HCl is FDA approved in the U.S. for use with blue light cystoscopy for the cystoscopic detection of Tat1 level nonmuscle invasive papillary bladder cancer (NMIBC). Hexaminolevulinate HCl staining is used with the KARL STORZ D-Light C Photodynamic Diagnostic (PDD) System to perform cystoscopy with the BLC™ (blue-light cystoscopy setting) (Mode 2) to enhance detection of bladder tumors. The mechanism is described as the selective accumulation of porphyrins in the rapidly dividing tumor cells. Photocure is pursuing FDA approval for use of Cysview® for post-treatment monitoring of bladder cancer patients. However, porphyrins, including those derived from hexaminolevulinate HCl, have a number of drawbacks, including a known incidence of allergic reactions and sensitization, and will not be suitable for all subjects.

Methods for treating bladder cancer include transurethral bladder tumor resection (TURBT), anti-cancer chemotherapy, radiation therapy and immunotherapy. Chemotherapy involves the disruption of cell replication or cell metabolism, and it remains one of the main treatment options for cancer. Chemotherapy, or chemotherapy combined with various types of radiation therapy can be effective, but there can be severe side effects. Because of the toxic side effects, many subjects receiving such chemotherapy and/or radiation therapy cannot successfully finish a complete chemotherapy regime. Advances in immunotherapy provide benefits relative to the older methods, and employ or activate cells of the immune system that exhibit cytotoxic activity against particular target cells.

One form of immunotherapy takes advantage of natural killer (NK) cells. NK cells are cytotoxic lymphocytes that constitute a major component of the innate immune system. NK cells generally represent about 10-15% of circulating lymphocytes. NK cells bind and kill targeted cells, including virus-infected cells and many malignant cells, non-specifically with regard to antigen and without prior immune sensitization. Herberman et al., Science 214:24 (1981). NK killing of targeted cells occurs by inducing cell lysis. NK cells employed for this purpose are isolated from the peripheral blood lymphocyte (“PBL”) fraction of blood from the subject, expanded in cell culture in order to obtain sufficient numbers of cells, and then re-infused into the subject. NK cells have been shown to be somewhat effective in both ex vivo therapy and in vivo treatment. NK-92 is a cytolytic cancer cell line which was discovered in the blood of a subject suffering from a non-Hodgkin's lymphoma and then immortalized ex vivo. NK-92 cells are derived from NK cells, but lack the major inhibitory receptors that are displayed by normal NK cells, while retaining the majority of the activating receptors. NK-92 cells do not, however, attack normal cells, nor do they elicit an unacceptable immune rejection response in humans. Characterization of the NK-92 cell line is disclosed, for example, by WO1998049268, U.S. 20040052770, and U.S. 20020068044. NK-92 cells have been evaluated as a therapeutic agent in the treatment of certain cancers, but it remains difficult to confirm, at an early stage of treatment, progress reducing the tumor size and mass.

Thus, there remains a longstanding need in the art for cost effective, efficient and relatively rapid methods of verifying or validating the effectiveness of individualized anti-bladder cancer therapy in a subject being treated for bladder cancer.

### SUMMARY OF THE INVENTION

Accordingly, the invention provides for a method of confirming the effectiveness of an anti-bladder cancer treatment in a subject diagnosed with a bladder cancer. In one embodiment, the method includes the steps of:

(a) infusing the bladder of the subject with a volume of a physiologically acceptable tumor selective dye or stain, in a physiologically acceptable solution or carrier, at a concentration effective to selectively stain tumor tissue in the lining of the bladder,

(b) detecting and measuring any bladder tumors stained by step (a) by conducting a cystoscopic procedure on the subject with a cystoscope, wherein the cystoscope comprises an endoscope for viewing the interior of the subject's bladder, and a system for illuminating the interior of the subject's bladder,

(c) treating the subject's bladder cancer,

(d) repeating steps (a) and (b) after step (c), as needed,

(e) comparing consecutive measurements of steps (c) and (d) to measure the degree of progression and effectiveness of the course of treatment of bladder cancer. The cystoscope for illuminating the interior of the subject's bladder comprises a light source, for example, such as a white light source, a blue light source, a laser illuminator and/or combinations thereof. For example, the white light source can be used simultaneously with the laser illuminator to photoactivate and visualize methylene blue and/or toluidine blue stained cancer tissue. The blue light source can be employed to photoactivate and visualize tumor tissue that has selectively taken up a dye that is metabolized to a photoactivatable compound within a tumor cell.

According to the invention, step (c) is repeated, as clinically determined for treating the subject's bladder cancer, and wherein steps (a) and (b) are repeated at an interval as determined to be clinically appropriate by the artisan, such as every two days, every week, every two weeks, every month, every two months, and/or every six months, until the subject's bladder cancer is in remission, or until a change of the treatment protocol is required.

The anti-bladder cancer therapy includes, for example, transurethral bladder tumor resection (TURBT), anti-cancer chemotherapy, radiation therapy and immunotherapy, administered separately, sequentially and/or in any combination. Preferably, the anti-bladder cancer therapy includes immunotherapy. In certain embodiments, the anti-bladder cancer therapy is optionally administered by an intravesical route and/or by a systemic route.

When the anti-bladder cancer therapy includes an immunotherapy, the immunotherapy is one or more of the following modalities: intravesical bacillus Calmette-Guérin (BCG) vaccine therapy, systemic immune checkpoint therapy, and NK cell therapy.

In particular embodiments, when the immunotherapy is NK cell therapy, the NK cells are allogenic and autologous, or are activated in vitro and optionally reinfused into the subject from whom the cells were obtained. When the NK cells are allogenic and autologous to the subject, the autologous NK cells are obtained by:

(a) isolating NK cells from the blood of the subject,

(b) expanding the isolated NK cells ex vivo in a suitable cell culture medium, and

(c) collecting the autologous NK cells expanded by step (b).

A further step includes, for example, infusing the collected autologous NK cells back into the subject.

In an alternative embodiment, the NK cells are NK-92 cells that are genetically modified. The genetically modified NK cells are, for example, modified to express at least one marker or antigen on the surface of the NK cells, where the marker provides targeted binding of the NK cells to the subject's bladder tumor. In a further embodiment, the autologous NK cells are activated in vitro by administering one or more NK activating cytokines to the subject.

Any of the above-described NK cells can be administered to a subject by infusion into the bloodstream of the subject and/or directly into or adjacent to a solid tumor or cancer to be treated.

In one embodiment, the tumor selective dye or stain is a dye that is converted to a photoactive porphyrin compound when selectively taken up by a tumor cell, such as hexaminolevulinate HCl.

In an alternative embodiment, the tumor selective dye or stain excludes any tumor selective dye or stain is a dye that is converted to a photoactive porphyrin compound when selectively taken up by a tumor cell.

In a further embodiment, the supravital dye or stain is selected from the group consisting of methylene blue (methylthionine chloride), toluidine blue (tolonium chloride), Evan's blue, and/or Gentian violet. Hexaminolevulinate HCl is another dye, that is FDA approved for diagnosis of particular types of bladder cancer.

### Definitions

In order to appreciate the present invention, the following terms are defined. Unless otherwise indicated, the terms listed below will be used and are intended to be defined as stated, unless otherwise indicated. Definitions for other terms can occur throughout the specification. It is intended that all singular terms also encompass the plural, active tense and past tense forms of a term, unless otherwise indicated.

An “effective amount” of an anti-bladder cancer treatment is an amount sufficient to effect beneficial or desired results, such as inhibiting, slowing or reversing the growth of the subject's bladder tumor. An effective amount of a tumor selective dye or stain is an amount or concentration in a range sufficient to selectively stain tumor cells, without creating false positive staining in adjacent normal tissues.

The phrase “consisting essentially of” means that the composition or method may include additional ingredients and/or steps, but only if the additional ingredients and/or steps do not materially alter the basic and novel characteristics of the claimed composition or method, i.e., the additional ingredient and/or step(s) would serve no purpose material to the claimed composition or method.

As understood in the art, the terms “tumor” and “cancer” are overlapping terms. A “tumor” is broadly considered to be a mass or growth found in an organism. A tumor cell is a cell derived from such a mass. A tumor can be benign or cancerous. A cancerous tumor, or “cancer” is a tissue growth that can spread out of control and invade other tissues, or in the case of blood cancers, overwhelm the circulatory system and/or seed cancers elsewhere in the body. A cancer cell is a cell derived from a cancer. For purposes of the invention, the terms “tumor cell” and “cancer cell” are used interchangeably, with the understanding that both refer to mammalian cells found in tumors or cancers or derived from and cultured from tumors or cancers, and that replicate abnormally, without the limits exhibited by differentiated mammalian cells.

It should also be understood that singular forms such as “a,” “an,” and “the” are used throughout this application for convenience, however, except where context or an explicit statement indicates otherwise, the singular forms are intended to include the plural. Further, it should be understood that every journal article, patent, patent application, publication, and the like that is mentioned herein is hereby incorporated by reference in its entirety and for all purposes.

All numerical ranges should be understood to include each and every numerical point within the numerical range, and should be interpreted as reciting each and every numerical point individually. The endpoints of all ranges directed to the same component or property are inclusive, and intended to be independently combinable.

As used herein, the term “about” means within 10% of the reported numerical value, preferably within 5% of the reported numerical value.

A “subject” or “patient” according to the invention is a human subject, such as a human patient with a tumor or cancer. In certain embodiments, the invention can also be applied in a veterinary practice to any subject, i.e., a vertebrate animal in need of such treatment. This could include, for example, mammal such as a non-human primate, a canine, a feline, a porcine, an equine, and/or any other mammal for which the inventive method is needed.

Various objects, features, aspects and advantages of the inventive subject matter will become more apparent from the following description of the drawing and from the detailed description of the invention.

### DETAILED DESCRIPTION OF THE INVENTION

Accordingly, the present invention provides for a method of measuring the progression and effectiveness of a course of treatment of bladder cancer in a subject diagnosed with a bladder cancer by the steps of:

(a) infusing the bladder of the subject with a volume of a physiologically acceptable tumor selective dye or stain, in a physiologically acceptable solution or carrier, at a concentration effective to selectively stain tumor tissue in the lining of the bladder,

(b) detecting and measuring any bladder tumors stained by step (a) by conducting a cystoscopic procedure on the subject with a cystoscope, wherein the cystoscope comprises an endoscope for viewing the interior of the subject's bladder, and a system for illuminating the interior of the subject's bladder,

(c) treating the subject's bladder cancer,

(d) repeating steps (a) and (b) after step (c),

(e) comparing consecutive measurements of steps (c) and (d) to measure the degree of progression and effectiveness of the course of treatment of bladder cancer.

According to the invention, step (c) is repeated, as clinically determined for treating the subject's bladder cancer, and wherein steps (a) and (b) are repeated at an interval as determined to be clinically appropriate by the artisan, such as every two days, every week, every two weeks, every month, every two months, and/or every six months, until the subject's bladder cancer is in remission, or until a change of the treatment protocol is required.

Cystoscopes and cystoscopy are well known in the art, and any suitable art-known cystoscopic instruments and techniques are readily adapted to the practice of the invention. Flexible cystoscopes only require that the subject receive local anesthesia, and are best suited for repeated testing according to the invention. Suitable instruments are available, for example, from Olympus Medical, Stryker, Advanced Endoscopy Devices (AED), Richard Wolf, Fujinon and others.

The system for illuminating the interior of the subject's bladder is a light source, for example, such as a white light source, a blue light source, a laser illuminator and/or combinations thereof. The light energy is delivered to the inside of the bladder via a suitable light conductive fiber or is delivered directly from an inserted miniaturized illuminator, e.g., a miniature light emitting diode source. In addition, the white light source can be used simultaneously, or alternatively, with a laser illuminator to photoactivate and visualize cancer tissue stained with methylene blue and/or toluidine blue or other appropriate stain or dye. The blue light source can be employed to photoactivate and visualize tumor tissue that has selectively taken up a dye that is a photoactivatable compound, or that is metabolized to form a photoactivatable compound, within a tumor cell.

The anti-bladder cancer therapy includes, for example, transurethral bladder tumor resection (TURBT), anti-cancer chemotherapy, radiation therapy and immunotherapy, administered separately, sequentially or in any combination.

The anti-bladder cancer therapy includes administering anti-cancer chemotherapeutic agents, either alone or in combination with immunotherapy, radiation therapy and/or surgical removal of cancerous bladder tissue, or surgical removal of tumors originating from a bladder cancer. Anticancer chemotherapeutic agents can be small molecule drugs or biologicals, such as monoclonal antibodies. According to the U.S. National Cancer Institute Web pages (www dot cancer dot gov) anti-bladder cancer chemotherapeutic agents approved by the US FDA include, but are not limited to: Atezolizumab, Bavencio® (Avelumab), Cisplatin, Doxorubicin Hydrochloride, Imfinzi® (Durvalumab) Keytruda® (Pembrolizumab), Opdivo® (Nivolumab) Pembrolizumab, Platinol® (Cisplatin) Platinol-AQ® (Cisplatin), Tecentriq®, and (Atezolizumab®) Thiotepa.

Anti-bladder cancer chemotherapeutic agents are often administered in combination and include, for example, a combined course of treatment with cisplatin and gemcitabine, a combined course of treatment with Carboplatin (Paraplatin) and gemcitabine, and an MVAC course of treatment. MVAC is a course of treatment with four drugs, separately administered: methotrexate, vinblastine, doxorubicin (Adriamycin®), and cisplatin. These and other chemotherapeutic agents can be administered by one or more separate routes of administration and with one or more dosing schedules. The MVAC is also optionally administered as dose-dense (DD) MVAC. This is art-known as an MVAC treatment with the administration schedule compressed into fewer days than employed for standard MVAC, in order to more effectively kill or inhibit rapidly replicating tumor cells.

In one particular embodiment, the anti-bladder cancer therapy is an immunotherapy. Immunotherapy can include, intravesical bacillus Calmette-Guérin (BCG) vaccine therapy. Immunotherapy can also include infusing expanded tumor-reactive CD4 helper and/or CD8+ T-lymphocytes obtainable from one or more sentinel or sentinel lymph nodes draining a tumor in the bladder or a metastasis arising from a tumor in the bladder, as described by U.S. Pat. No. 8,101,173. Other immunotherapies according to the invention include systemic immune checkpoint therapy, e.g., administering Nivolumab 240 mg IV q2wk infused over 60 min until disease progression or unacceptable toxicity, Durvalumab 10 mg/kg IV q2wk infused over 60 min until disease progression or unacceptable toxicity, Avelumab 10 mg/kg infused over 60 min until disease progression or unacceptable toxicity [17], and natural killer (NK) cell therapy.

In certain particular embodiments, the immunotherapy is by administration of therapeutic NK cells. Depending on the clinical requirements, the NK cells are allogenic and autologous, or are activated in vitro and reinfused into the subject. When the NK cells are allogenic and autologous to the subject, the autologous NK cells are obtained by:

(a) isolating NK cells from the blood of the subject,

(b) expanding the isolated NK cells ex vivo in a suitable cell culture medium, and

(c) collecting the autologous NK cells expanded by step (b), and these collected autologous NK cells are infused back into the subject as needed.

See, for example, Torelli et al., 1015, Blood Transfus 13; 464-71 DOI10.2450/2015.0231-14, describing a two-step immunomagnetic procedure consisting of CD3+ T-cell depletion followed by CD56+ cell positive selection to obtain NK cells.

In a further embodiment, the autologous NK cells are activated in vitro by administering one or more NK activating cytokines, such as IL-15 to the subject. In a still further embodiment, the NK cells are genetically modified NK-92 cells that include, for example, NK cells modified to express at least one marker or antigen on the surface of the NK cells, where the marker provides targeted binding of the NK-92 cells to the subject's bladder tumor cells, and/or permits visualization or monitoring of the NK cells in vivo.

In a more particular embodiment, the genetically engineered allogenic NK cell is an NK-92 derivative (i.e., a genetically modified NK-92 cell) that has reduced or abolished expression of at least one killer cell immunoglobulin-like receptor (KIR), which will render such cells constitutively activated (via lack of or reduced inhibition). These are described, for example, by WO2017100709. Therefore, suitable modified NK cells may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and/or KIR3DS1. Such modified NK-92 cells may be prepared, for example, using silencing protocols, CIRSPR-CAS genome editing, or knock-out or knock-down protocols well known in the art. Alternatively, such modified NK-92 cells may also be commercially obtained from NantKwest (see the Nantkwest dot com website) as aNK cells (activated natural killer cells). Such cells may then be further modified to express one or more ligands for one or more inhibitory receptors of the NK cells of the host organism.

Although NK-92 cells retain almost all of the activating receptors and cytolytic pathways associated with NK cells, they do not express CDI6 on their cell surfaces. CD16 is an Fc receptor which recognizes and binds to the Fc portion of an antibody to activate NK cells for antibody-dependent cellular cytotoxicity (ADCC). Due to the absence of CD16 receptors, NK-92 cells are unable to lyse target cells via the ADCC mechanism. Thus, in another aspect of the invention, the genetically engineered NK cell may also be an NK-92 derivative that is modified to express a high-affinity Fcγ receptor (e.g., CD16, V158) as described by WO2016160602. Sequences for high-affinity variants of the Fcγ receptor are well known in the art, and all methods of generating and expression are deemed suitable for use herein. Without meaning to be bound by any theory or hypothesis as to the operation of these receptors, expression of such receptors is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM).

Advantageously, such anti-neoepitope antibodies are commercially available and can be used in conjunction with the NK-92 derivative cells (e.g., bound to the Fcγ receptor). Alternatively, such cells may also be commercially obtained from NantKwest as haNK cells (high-affinity natural killer cells). Such cells may then be further modified to express one or more ligands for one or more inhibitory receptors of the NK cells of the host organism.

In a further aspect of the invention, the genetically engineered NK cells may also be genetically engineered to express a chimeric antigen receptor (CAR), as described by WO 2016160621. In especially preferred aspects, the chimeric antigen receptor will have a scFv portion or other ectodomain with binding specificity against a tumor associated antigen, a tumor specific antigen, and a cancer neoepitope. As noted before, there are numerous art known methods of genetically engineering an NK cell to express such chimeric T-cell receptor, and all such art known methods are deemed suitable for use herein. Alternatively, such cells may also be commercially obtained from NantKwest as taNK cells (‘target-activated natural killer cells’). Such cells may then be further modified to express one or more ligands for one or more inhibitory receptors of the NK cells of the host organism.

Where the NK cells are engineered to have affinity for a cancer associated antigen or for an antibody with specificity for a cancer associated antigen, it is contemplated that all known cancer associated antigens are considered appropriate for use. For example, cancer associated antigens include CEA, MUC-1, CYPB1, etc. Likewise, where the cells are engineered to have affinity towards a cancer specific antigen or antibody with specificity towards a cancer specific antigen, it is contemplated that all known cancer specific antigens are considered appropriate for use. For example, cancer specific antigens include PSA, Her-2, PSA, brachyury, etc. Where the cells are engineered to have affinity towards a cancer neoepitope or antibody with specificity towards a cancer neoepitope, it is contemplated that all known methods of identifying neoepitopes will lead to suitable targets. For example, neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous comparison of the so obtained omics information. So identified neoepitopes can then be further filtered for a match to the patient's HLA type to increase likelihood of antigen presentation of the neoepitope. Most preferably, such matching can be done in silico.

In further contemplated aspects of the invention, allogenic NK cells may also be obtained from a cell bank or cell culture, where the allogenic NK cells are preferably (but not necessarily) HLA matched to a depth of at least two, and more typically at least four digits. Where such cells are not available or otherwise not desired, it is contemplated that allogenic NK cells may also be grown from various precursor cells as is described, for example, in WO2017070337 or U.S. 20140186319.

The route of administration, dosing and frequency of the anti-bladder cancer chemotherapy and/or immunotherapy is selected by the artisan as appropriate for the therapeutic modality and clinical condition of the subject, and chemotherapy or immunotherapy can be delivered by art-known alternative art known routes of administration, as appropriate. Available routes of administration include subcutaneous injection, intramuscular injection, intravenous injection, intra-arterial injection, oral administration, intravesicular administration or infusion, direct injection into the bladder tumor tissue via appropriate transurethral instrumentation into the bladder, and other parenteral routes.

### Dyes and Stains

The dye or stain is dissolved in a physiologically acceptable solution or carrier. This is generally an iso-osmotic saline in water solution, at 0.9% saline, and/or a nontoxic, iso-osmotic buffer solution, such as a phosphate buffer or other physiologically acceptable buffer systems, where pH control is necessary to optimize selective tissue staining.

Generally, the dye or stain is a supravital dye selected from the group consisting of methylene blue (methylthionine chloride), toluidine blue (tolonium chloride), Evan's blue, hexaminolevulinate HCl and/or Gentian violet. Preferably, the supravital dye is methylene blue. In certain embodiments, the dye is a mixture designed to enhance contrast. For example, a mixture of methylene blue, malachite, and eosin as described by Riaz et al. (SpringerPlus 2013, 2:95) for selective staining of gastrointestinal tumors.

For direct cystoscopic visualization of bladder tumors, methylene blue is infused into the bladder of a subject, e.g., via a Foley catheter, in a concentration of from about 0.5% to about 1.8% methylene blue in physiological saline, but generally 1% methylene blue is used. After about five minutes, the methylene blue solution is drained, and the bladder washed with physiological saline, at least three times, as described by Gil. Alternatively, the bladder is washed with a 1% lactic acid solution, as employed by Riaz et al. Id. supra, to improve removal of nonspecific staining for oral cancers. A standard cystoscope is then used to visualize the inner bladder wall for blue stained tissue, that highlights those tumors visible on or within the bladder surface.

For cystoscopic visualization of photosensitized methylene blue, the methylene blue is administered to the bladder wall or adjacent to the bladder tumor in a physiologically acceptable solution in a concentration from about 0.0075% to about 0.02%, and stained tissue is illuminated with light energy at approximately 660 nm. For visualization of photosensitized toluidine blue, the toluidine blue is administered to the bladder wall or adjacent to the bladder tumor in a tumor in a physiologically acceptable solution in a concentration from about 0.0075% to about 0.02%, and stained tissue is illuminated with light energy at approximately 660 nm. The light energy is preferably delivered by a suitable laser illuminator, e.g., conducted into the bladder via a fiber optic system or directly from a laser instrument inserted into the bladder.

In different embodiments, porphyrin-based systems, such as hexaminolevulinate HCl, may also be employed according to the invention. Tumor cells selectively take up hexaminolevulinate HCl and convert the hexaminolevulinate HCl to several photoactivatable porphyrin compounds. Alternatively, hexaminolevulinate HCl and/or other dyes that selectively stain tumor cells with porphyrin compounds, are expressly excluded from the practice of the present invention.

When a subject is diagnosed with a bladder tumor, the bladder wall is visualized by the appropriate stain or dye, and the area, intensity and anatomical distribution of the staining is measured and recorded. Measurement methods include visual grading of the tumor by the artisan, photometric measurement of fluorescent light from the stained tissues (i.e., fluorometric intensity) and/or by tracking the progress of anti-bladder cancer therapy by recording a photographic record of the subject's pre-treatment bladder wall, to compare to photographic records of subsequence post-treatment staining of the subject's bladder wall.

Once the clinically appropriate anti-bladder cancer treatment is commenced, the subject is periodically retested to measure the progress and results of the selected anti-bladder cancer therapy. The frequency of testing is determined by the artisan in view of the clinical status of the subject, and is continued until the maximal benefits of the treatment are achieved. Depending on the judgement of the artisan, the testing can be conducted every two days, every week, every two weeks, every month, every two months, and/or every six months, until the goals of the anti-tumor bladder treatment are reached, or until a change of the treatment protocol may be required.

### INCORPORATION BY REFERENCE

All publications, patents, and patent applications recited herein are incorporated by reference to the same extent as if each individual publication, patent, or patent application are specifically and individually incorporated by reference. Where a definition or use of a term in an incorporated publication, patent, or patent application is inconsistent or contrary to the definition of that term provided herein, the definition of that term provided herein applies and the definition of that term in the reference does not apply.

We claim:

1. A method of measuring the progression and effectiveness of a course of treatment of bladder cancer in a subject diagnosed with a bladder cancer, comprising,
(a) infusing the bladder of the subject with a volume of a physiologically acceptable tumor selective dye or stain, in a physiologically acceptable solution or carrier, at a concentration effective to selectively stain tumor tissue in the lining of the bladder,
(b) detecting and measuring any bladder tumors stained by step (a) by conducting a cystoscopic procedure on the subject with a cystoscope, wherein the cystoscope comprises an endoscope for viewing the interior of the subject's bladder, and a system for illuminating the interior of the subject's bladder,
(c) treating the subject's bladder cancer,
(d) repeating steps (a) and (b) after step (c),
(e) comparing consecutive measurements of steps (c) and (d) to measure the degree of progression and effectiveness of the course of treatment of bladder cancer.

2. The method of claim 1, wherein the anti-bladder cancer therapy is selected from the group consisting of transurethral bladder tumor resection (TURBT), anti-cancer chemotherapy, radiation therapy and immunotherapy.

3. The method of claim 2, wherein the anti-bladder cancer therapy is selected from the group consisting of anti-cancer chemotherapy, radiation therapy and immunotherapy.

4. The method of claim 3, wherein the anti-bladder cancer therapy is anti-cancer chemotherapy and/or immunotherapy.

5. The method of claim 1, wherein step (c) is repeated, as clinically determined for treating the subject's bladder cancer, and wherein steps (a) and (b) are repeated at an interval selected from the group consisting of every two days, every week, every two weeks, every month, every two months, and every six months, until the subject's bladder cancer is in remission, or until a change of the treatment protocol is required.

6. The method of claim 4, wherein the anti-bladder cancer therapy is administered by an intravesical route or by a systemic route.

7. The method of claim 4, wherein the anti-bladder cancer therapy is an immunotherapy.

8. The method of claim 7, wherein the immunotherapy is selected from the group consisting of intravesical bacillus Calmette-Guérin (BCG) vaccine therapy, systemic immune checkpoint therapy, and natural killer (NK) cell therapy.

9. The method of claim 8, wherein the NK cells are allogenic and autologous, or are activated in vitro and reinfused into the subject.

10. The method of claim 8, wherein the NK cells are allogenic and autologous to the subject,
wherein the autologous NK cells are obtained by
(a) isolating NK cells from the blood of the subject,
(b) expanding the isolated NK cells ex vivo in a suitable cell culture medium, and
(c) collecting the autologous NK cells expanded by step (b).

11. The method of claim 10, further comprising a step of infusing the collected autologous NK cells back into the subject.

12. The method of claim 8, wherein the NK cells are genetically modified NK-92 cells.

13. The method of claim 8, wherein the NK cells are modified to express at least one marker or antigen on the surface of the NK cells, where the marker provides targeted binding of the NK cells to the subject's bladder tumor.

14. The method of claim 8, wherein the NK cells are administered by infusion into the bloodstream of the subject.

15. The method of claim 8, wherein autologous NK cells are activated in vitro in vitro by administering one or more NK activating cytokines to the subject.

16. The method of claim 1, wherein the tumor selective dye or stain is selected from the group consisting of methylene blue (methylthionine chloride), toluidine blue (tolonium chloride), and Evan's blue, and/or Gentian violet.

17. The method of claim 16, wherein the supravital dye is methylene blue.

18. The method of claim 1, wherein the tumor selective dye or stain is converted to a photoactive porphyrin compound when taken up by a tumor cell.

19. The method of claim 18, wherein the tumor selective dye or stain is hexaminolevulinate HCl.

20. The method of claim 1, wherein system for illuminating the interior of the subject's bladder comprises a light source selected from the group consisting of a white light source, a blue light source, a laser illuminator and combinations thereof.
